(19) United States
(12) Patent Application Publication
(10) Pub. No.: US 2023/0142101 A1
(43) Pub. Date: May 11, 2023

(54) LIFELOG PROVIDING SYSTEM AND LIFELOG PROVIDING METHOD

(71) Applicant: PANASONIC INTELLECTUAL PROPERTY MANAGEMENT CO., LTD., Osaka (JP)

(72) Inventors: Sonoko HIRASAWA, Kanagawa (JP); Takeshi FUJIMATSU, Kanagawa (JP)

(73) Assignee: PANASONIC INTELLECTUAL PROPERTY MANAGEMENT CO., LTD., Osaka (JP)

(21) Appl. No.: 17/913,360

(22) PCT Filed: Feb. 11, 2021
(86) PCT No.: PCT/JP2021/005125

§ 371 Date: Sep. 21, 2022

(30) Foreign Application Priority Data
Mar. 27, 2020 (JP) 2020-057915

(51) Int. Cl. G06T 5/50; G06V 20/40; G06V 40/10; G06V 40/16; G06V 40/20; G06F 3/0482

(52) U.S. Cl. CPC G06T 5/50; G06V 20/44; G06V 20/46; G06V 40/103; G06V 40/174; G06V 40/20; G06F 3/0482; G06T 2207/20221

(57) ABSTRACT

Provided is a lifelog providing system for providing a user with a lifelog of a child that enables the user to systematically recognize a level of growth of the child. The system is configured to detect a specific event related to a level of growth of the child in images captured by a camera, by using image recognition; extract a scene image including the detected specific event, from the images captured by the camera; and generate, as a lifelog, a growth map in which a thumbnail of the scene image is overlaid on a reference map image including a timeline of child growth and an indicator showing a normal pace of growth for each specific event, such that the thumbnail of the scene image is located at a point in the reference map image corresponding to date and time of the detection of the specific event.

### TECHNICAL FIELD

The present disclosure relates to a lifelog providing system and a lifelog providing method for providing a user with an image of a child captured by a camera in a childcare facility, as a lifelog.

### BACKGROUND ART

In recent years, as the number of double-income families is growing, an increased number of parents send their children to daycare earlier than before, and some start at six months of age or earlier, resulting in that parents have less opportunity to see scenes of “specific events indicative of the growth of children” i.e., children's developmental milestones. As a result, many parents feel frustrated about problems associated with daycare use. For example, some parents refrain from sending a baby to daycare at an early age, and other parents regret having failed to see memorable scenes of their children's developmental milestones later. Moreover, parents using daycare are only able to know how a child grows through reports from nurses in daycare. Therefore, there is a need for technologies to eliminate the parents' frustration.

Known technologies to address this issue include a system capable of analyzing images of a child captured by cameras, and extracting, from the captured images, images that are recognized to show “impressive scenes” for parents, such as an image showing the child's smile on a specific day, an image showing how the baby stood along for the first time, and an image showing how the baby took his or her first steps (Patent Document 1). This system enables parents to watch children's impressive scenes which the parents could not have viewed directly, thereby decreasing the parents' frustration.

### PRIOR ART DOCUMENT(S)

### Patent Document(s)

Patent Document 1: JP2019-125870A

### SUMMARY OF THE INVENTION

### Task to be Accomplished by the Invention

The above-described system of the prior art can present captured images showing children's impressive scenes to parents. However, since whether or not a scene is impressive to parents is determined based on the subjective view of an individual parent, the system cannot always select images that are desirable to parents. Generally, parents who use such a system are only able to know how their child grows through reports from nurses in daycare. Thus, when images of a child in daycare are used as a lifelog of the child for the parents, such images need to recognizably show how the child grows. In particular, such lifelog images need to enable parents to systematically recognize their children's levels of growth based on evaluation bases common to any parent.

The present disclosure has been made in view of the problem of the prior art, and a primary object of the present disclosure is to provide a lifelog providing system and a lifelog providing method which can provide a user with a lifelog of a child that enables the user to systematically recognize a level of growth of the child.

### Means to Accomplish the Task

An aspect of the present invention provides a lifelog providing system in which at least one data processing device performs operations for providing a user with an image of a child captured by a camera in a facility, as a lifelog, wherein the at least one data processing device is configured to: detect a specific event related to a level of growth of the child in images captured by the camera, by performing an image recognition operation; extract a scene image including the detected specific event, from the images captured by the camera; and generate, as a lifelog, a growth map in which a thumbnail of the scene image is overlaid on a reference map image, the reference map image including at least a timeline of child growth and an indicator showing a normal pace of growth of children for each specific event, such that the thumbnail of the scene image is located at a point in the reference map image corresponding to date and time of the detection of the specific event.

Another aspect of the present invention provides a lifelog providing method in which at least one data processing device performs operations for providing a user with an image of a child captured by a camera in a facility, as a lifelog, wherein the at least one data processing device performs operations of: detecting a specific event related to a level of growth of the child in images captured by the camera, by performing an image recognition operation; extracting a scene image including the detected specific event, from the images captured by the camera; and generating, as a lifelog, a growth map in which a thumbnail of the scene image is overlaid on a reference map image, the reference map image including at least a timeline of child growth and an indicator showing a normal pace of growth of children for each specific event, such that the thumbnail of the scene image is located at a point in the reference map image corresponding to date and time of the detection of the specific event.

### Effect of the Invention

According to the present disclosure, with use of an indicator showing a normal pace of growth of children, users such as parents can systematically determine their children's levels of growth based on objective evaluation bases, which are independent from the subjective view of an individual. This configuration also enables parents and facility staff to recognize children's levels of growth based on their common evaluation bases. Accordingly, it is possible to provide a user with a lifelog of a child that enables the user to systematically recognize a level of growth of the child.

### BRIEF DESCRIPTION OF THE DRAWINGS

FIG. 1 is a diagram showing an overall configuration of a lifelog providing system according to one embodiment of the present disclosure;

FIG. 2 is an explanatory diagram showing primary components of the lifelog providing system;

FIG. 3 is an explanatory diagram showing screen transitions on a user terminal 5;

FIG. 4 is an explanatory diagram showing a growth map screen displayed on the user terminal 5;

FIG. 5 is a block diagram showing schematic configurations of an edge computer 3 and a cloud computer 4;

FIG. 6 is an explanatory diagram showing management information processed by the cloud computer 4;

FIG. 7 is a flow chart showing a procedure of processing operations performed by the edge computer 3;

FIG. 8 is a flow chart showing a procedure of a face verification operation performed at the cloud computer 4; and

FIG. 9 is a flow chart showing a procedure of a log-in operation, a growth map generation operation, and a distribution operation performed at the cloud computer 4.

### DESCRIPTION OF THE PREFERRED EMBODIMENT(S)

A first aspect of the present invention made to achieve the above-described object is a lifelog providing system in which at least one data processing device performs operations for providing a user with an image of a child captured by a camera in a facility, as a lifelog, wherein the at least one data processing device is configured to: detect a specific event related to a level of growth of the child in images captured by the camera, by performing an image recognition operation; extract a scene image including the detected specific event, from the images captured by the camera; and generate, as a lifelog, a growth map in which a thumbnail of the scene image is overlaid on a reference map image, the reference map image including at least a timeline of child growth and an indicator showing a normal pace of growth of children for each specific event, such that the thumbnail of the scene image is located at a point in the reference map image corresponding to date and time of the detection of the specific event.

According to this configuration, with use of an indicator showing a normal pace of growth of children, users such as parents can systematically determine their children's levels of growth based on objective evaluation bases, which are independent from the subjective view of an individual. This configuration also enables parents and facility staff to recognize children's levels of growth based on their common evaluation bases. Accordingly, it is possible to provide a user with a lifelog of a child that enables the user to systematically recognize a level of growth of the child.

A second aspect of the present invention is the lifelog providing system of the first aspect, further comprising: an edge computer installed in the facility; and a cloud computer connected to the edge computing device via a network; wherein the at least one processing device comprises a first processing device provided in the edge computer and a second processing device provided in the cloud computer, wherein the first processing device performs operations for detecting the specific event and extracting the scene image, and transmits the scene image to the cloud computer, and wherein the second processing device generates the growth map based on the scene image received from the edge computer, and distributes the growth map to a user device.

This configuration can reduce the amount of data transmitted from the edge computer to the cloud computer, thereby decreasing the communication load on a communication link

A third aspect of the present invention is the lifelog providing system of the first aspect, wherein the at least one processing device is configured to detect the specific event by performing the image recognition operation, wherein the image recognition operation comprises at least one of a body frame detection operation, an action recognition operation, and a facial expression estimation operation.

This configuration enables accurate detection of a specific event.

A fourth aspect of the present invention is the lifelog providing system of the first aspect, wherein the at least one processing device is configured to, upon detecting a user's operation to select one of thumbnails in the growth map, cause a user device to display time information indicating date and time of occurrence of the specific event corresponding to the selected thumbnail.

This configuration enables a user to easily confirm date and time of occurrence of a specific event.

A fifth aspect of the present invention is the lifelog providing system of the first aspect, wherein the at least one processing device is configured to, upon detecting a user's operation to select one of thumbnails in the growth map, cause a user device to reproduce the scene image corresponding to the selected thumbnail.

This configuration enables a user to easily view a scene image related to a specific event of the user's interest.

A sixth aspect of the present invention is the lifelog providing system of the first aspect, wherein the at least one processing device is configured to, upon detecting a user's add-to-favorite operation, add a selected specific event to favorites.

This configuration enables a user to add a specific event of the user's interest to favorites, thereby allowing the user to repeatedly view the specific event with ease.

A seventh aspect of the present invention is the lifelog providing system of the first aspect, wherein the at least one processing device is configured to, upon detecting a user's operation to view favorites, cause a user device to display a list of information on specific events in favorites.

This configuration enables a user to easily confirm information on specific events in favorites. Examples of items included in information on a list of specific events include, for each event, the name of a specific event, the date and time of occurrence of the specific event, the age of a subject child in months (number of months after birth).

An eighth aspect of the present invention is a lifelog providing method in which at least one data processing device performs operations for providing a user with an image of a child captured by a camera in a facility, as a lifelog, wherein the at least one data processing device performs operations of: detecting a specific event related to a level of growth of the child in images captured by the camera, by performing an image recognition operation; extracting a scene image including the detected specific event, from the images captured by the camera; and generating, as a lifelog, a growth map in which a thumbnail of the scene image is overlaid on a reference map image, the reference map image including at least a timeline of child growth and an indicator showing a normal pace of growth of children for each specific event, such that the thumbnail of the scene image is located at a point in the reference map image corresponding to date and time of the detection of the specific event.

According to this configuration, it is possible to provide a user with a lifelog of a child that enables the user to systematically recognize a level of growth of the child in the same manner as the first aspect.

Embodiments of the present disclosure will be described below with reference to the drawings.

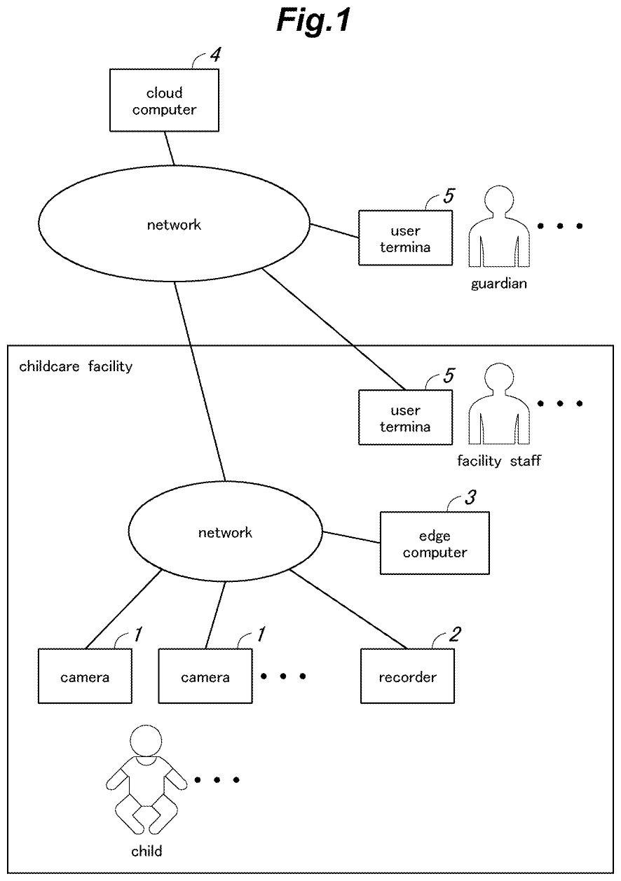

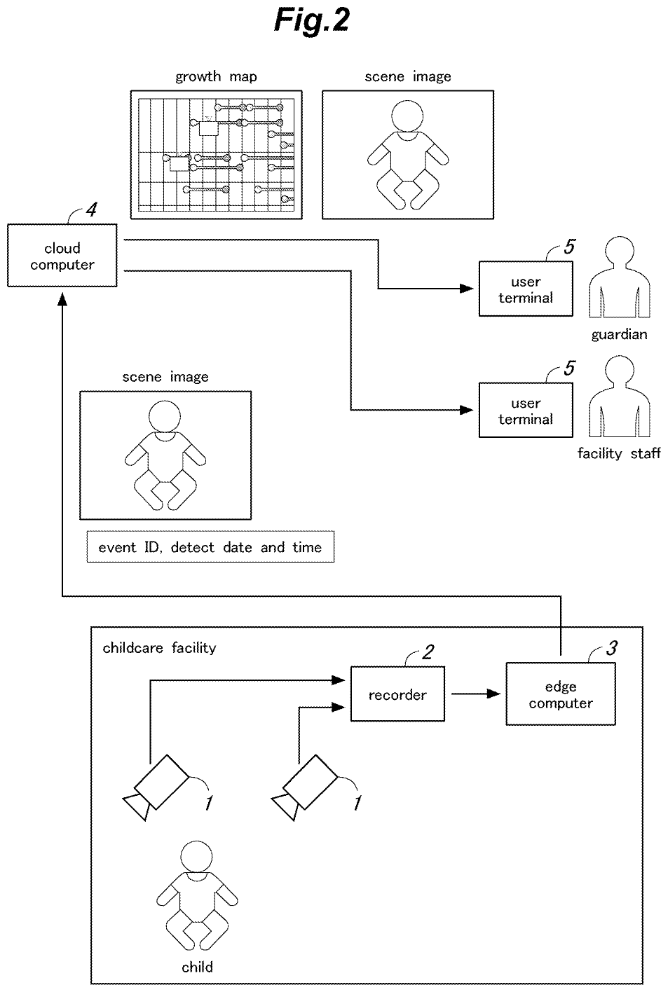

FIG. 1 is a diagram showing an overall configuration of a lifelog providing system according to one embodiment of the present disclosure. FIG. 2 is an explanatory diagram showing primary components of the lifelog providing system.

The lifelog providing system is configured to provide users with captured images of a child (baby and toddler) put in a childcare facility such as a daycare facility, as a lifelog, and examples of users of the system include parents (typically parents who send their child to daycare) and facility staff such as nurses engaged in childcare work at a childcare facility. The lifelog providing system includes cameras 1, a recorder 2, an edge computer 3, a cloud computer 4, and a user terminal 5 (user device).

The cameras 1, the recorder 2, and the edge computer 3 are installed in a child care facility. The cameras 1, the recorder 2, and the edge computer 3 are connected to each other via a network such as a LAN. The edge computer 3, the cloud computer 4, and the user terminal 5 are connected to each other via a network such as the Internet.

Each camera 1 captures images of a certain area inside of the child care facility. The cameras 1 constantly capture daily-life scenes of children in the childcare facility.

The recorder 2 stores (records) images captured by the cameras 1.

The edge computer 3 acquires images captured by the cameras 1 from the recorder 2, detects a child's specific event related to a level of growth (such as a child's developmental milestone), in the captured images by performing an image recognition operation, extracts, based on a detection result, a scene image including the detected specific event from the images captured by the cameras, and transmits the scene image and related information records (such as an event ID of the detected specific event and detection date and time) to the cloud computer 4. As used herein, the term “specific event” refers to an event that is one of various events occurred in children (acts, facial expressions, and physical states), and that can be a basis (evaluation item) to determine a level of growth of a child.

The cloud computer 4 identifies the child in the scene image received from the edge computer 3 through face verification, and associates the scene image with information on the child that has been previously registered. The cloud computer 4 also generates a growth map that visualizes levels of growth (degrees of growth) of children. The cloud computer 4 manages a log-in to the system from the user terminal 5, and distributes the growth map and the scene image of a child related to a user, as a lifelog of the child, to the user terminal 5.

The user terminal 5 may be a personal computer (PC) or a smartphone. A guardian (such as a parent) or a facility staff member (such as a nurse in daycare) operates the user terminal 5 as a user. In the present embodiment, the user terminal 5 displays a growth map and a scene image distributed as a lifelog from the cloud computer 4. As a result, a user such as a guardian or facility staff for a child can view the growth map and a scene image of the child.

In the present embodiment, the system is configured to include two data processing devices; that is, the edge computer 3 and the cloud computer 4. However, in other embodiments, the system may include a single data processing device that implements both the functions of the edge computer 3 and the cloud computer 4. In other words, the system may be configured to include only one of the edge computer 3 and the cloud computer 4.

In the present embodiment, the system is configured to extract a scene image including a specific event from images captured by the cameras 1 installed in a childcare facility. In other embodiments, the system may extract a scene image from images captured by any other device (such as a smartphone) at a different place (such as a park where a child and a parent have visited).

In the present embodiment, the edge computer 3 detects a specific event and extracts a scene image (moving image) including the specific event, from images recorded in the recorder 2. In other embodiments, the system may be configured such that a facility staff member or any other guardian operates a terminal to select (extract) a scene image including a specific event. In some cases, the edge computer 3 may extract candidates for a scene image, so that a facility staff member can select one of the candidates as an extracted scene image.

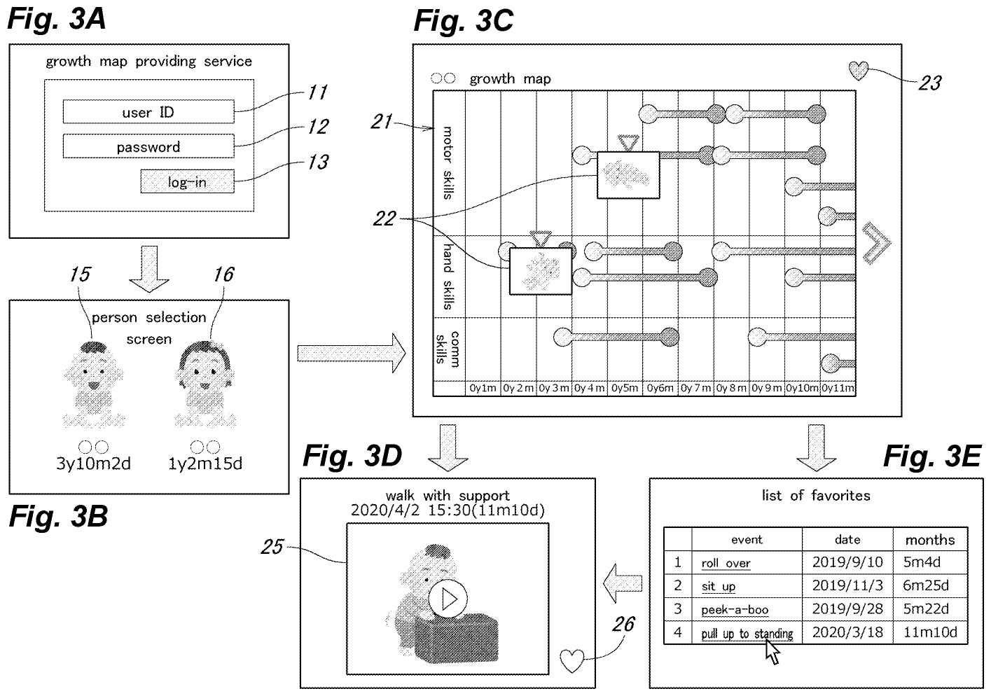

Next, screens displayed on a user terminal 5 will be described. FIG. 3 is an explanatory diagram showing screen transitions on the user terminal 5.

Upon accessing the cloud computer 4, the user terminal 5 first displays a log-in screen shown in FIG. 3A. When a user enters the user's user ID and password at entry fields 11 and 12 and operates a log-in button 13 in the log-in screen, the screen transitions to a person selection screen shown in FIG. 3B.

The person selection screen shown in FIG. 3B includes person selection menus 15 and 16, each for a corresponding one of the registered children. The person selection screen further indicates, for each of the menus 15 and 16, a person's image, name, and age in months. When a user operates a selection menu in the person selection screen to thereby select one of the person selection menus 15 and 16, the screen transitions to a growth map screen shown in FIG. 3C.

The user terminal 5 displays the person selection screen when a logged-in user is a guardian who puts a plurality of children to a childcare facility, or a facility staff member. When a logged-in user is a guardian who puts only one child to a childcare facility, the user terminal 5 skips the display of the person selection screen. Moreover, when a logged-in user is a guardian such as a parent, the user terminal 5 displays the guardian's child or children. When a logged-in user is a facility staff member such as a nurse in daycare, the user terminal 5 displays the child or children the staff member is responsible for.

The growth map screen shown in FIG. 3C indicates a growth map 21 for the child selected by a user. The growth map 21 includes thumbnails 22 of scene images, each scene image showing a corresponding motion of the child designated as a specific event. When the user operates a thumbnail in the growth map screen to thereby select one of the thumbnails 22, the screen transitions to a moving image reproduction screen shown in FIG. 3D. The growth map screen includes a view-favorite mark 23. When a user operates the view-favorite mark 23, the screen transitions to a favorite list screen shown in FIG. 3E.

The moving image reproduction screen shown in FIG. 3D includes a moving image viewer 25. The moving image viewer 25 reproduces a scene image (moving image) related to a specific event corresponding to the thumbnail 22 selected by the user in the growth map. The moving image reproduction screen indicates the name of a specific event, the date and time of occurrence of the specific event (shooting date and time), the child's age in months at the time of the detection of the specific event (shooting time point). Moreover, the moving image reproduction screen indicates an add-to-favorite mark 26. A user can operate the add-to-favorite mark 26 to thereby add the selected specific event to favorites.

The favorite list screen shown in FIG. 3E indicates information on a list of specific events added to favorites, selected from the specific events that have been detected in the images of a subject child. Specifically, the favorite list screen indicates, for each event, the name of a specific event (“event”), the date and time of the detection of the specific event (“date of occurrence”), and the age of the child in months at the time of the detection of the specific event (“age in months”). When a user operates on the favorite list screen to select the name of a specific event, the screen transitions to the moving image reproduction screen shown in FIG. 3D.

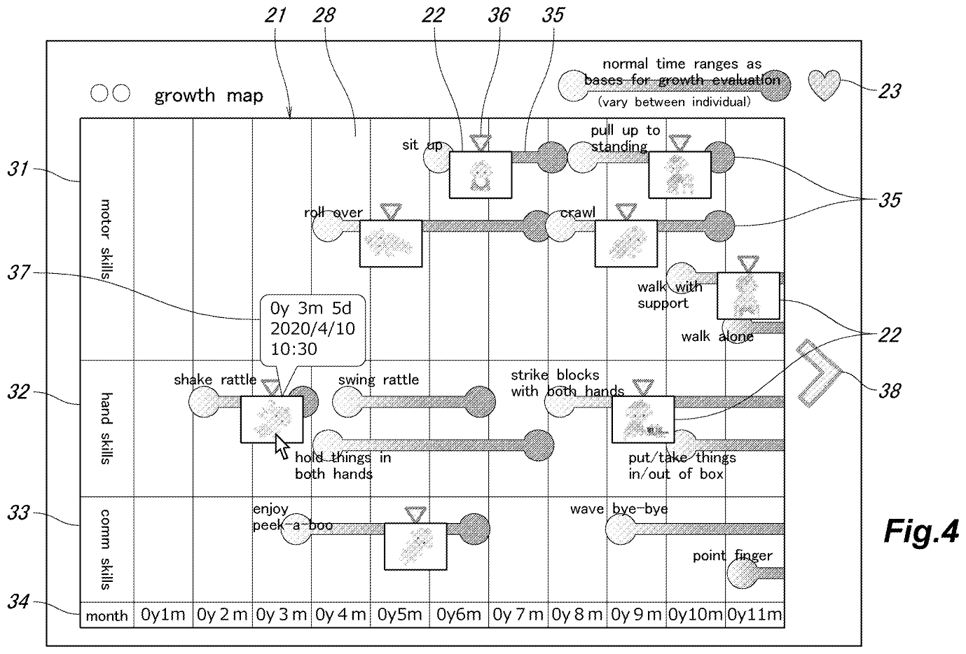

Next, a growth map screen displayed on the user terminal 5 will be described. FIG. 4 is an explanatory diagram showing the growth map screen displayed on the user terminal 5.

The growth map screen shows a growth map 21 that visualizes a level of growth (degree of growth) of a child. The growth map 21 includes thumbnails 22 of scene images overlaid on a map image 28, each scene image showing a corresponding motion of the child as a specific event.

The map image 28 includes items of categories of specific events that can be evaluation bases to determine a level of growth of a child, which items consist of the item 31 (“motor skills”) for specific events related to the motor development, the item 32 (“hand skills”) for specific events related to the dexterity development, and the item 33 (“comm. skills”) for specific events related to the mental development (development of social-emotional-verbal skills).

In the example shown in FIG. 4, specific events in the item (“motor skills”) related to the motor development include sitting up, pulling up to standing, rolling over, crawling, walking with support, and walking alone. Specific events in the item (“hand skills”) related to the dexterity development include shaking the rattle, swinging the rattle, striking things (blocks) with both hands, holding things in both hands, and putting and taking things in and out of the box. Specific events in the item (“comm. skills”) related to the mental development (development of social-emotional-verbal skills) include enjoying peek-a-boo, waving bye-bye, and pointing a finger.

In the present embodiment, the “growth” is growth of physical abilities and mental abilities (i.e., the development of physical skills and mental skills). However, the “growth” may include physical growth such as an increase in height or weight.

The map image 28 includes column footers 34 each for corresponding months of age. The respective column footers 34 can be a time base for each event related to a level of growth.

The map image 28 includes normal time range marks 35 (indicators for pace of growth). Each normal range mark 35 represents a normal range of time in which a corresponding specific event occurs (i.e. children achieve a certain developmental milestone), that can be evaluation bases for the child's growth.

The map image 28 further includes event detection marks 36, each event detection mark indicating the detection time point (shooting time point) in an age of a child in months at which a corresponding specific event is detected. Thus, each event detection mark 36 is indicated at a location for the detection time point (shooting time point) of a corresponding specific event. Indicated adjacent to each event detection mark 36 is a thumbnail 22 of a corresponding scene image. Thus, the map image enables users (i.e., guardians such as parents and facility staff members such as nurses in daycare) to recognize levels of growth of a child based on comparison between detection time points and corresponding normal ranges of time (normal paces of growth), so that the users can easily confirm whether or not the child is normally growing. From the map image, users can also acquire useful information for future child rearing and childcare, which means that the users can do appropriate practice of child rearing and childcare according to the level of growth of the child.

When a specific event is detected at a time point that is out of a corresponding normal range of time, an event detection mark 36 and a thumbnail 22 for the specific event are indicated on the left or right side of a corresponding normal time range mark 35. When a user operates the map to select one of the thumbnails 22, the screen transitions to a moving image reproduction screen shown in FIG. 3D.

When a user causes a pointer to move over a thumbnail 22 (performs a mouse over operation), a balloon 37 appears in the screen. Indicated in the balloon 37 is a time stamp for a corresponding specific event; that is, time information indicating date and time of occurrence of the specific event.

The growth map screen includes a scroll button 38. By operating the scroll button 38, a user can scroll the growth map 21 horizontally, which enables the growth map 21 including a longer timeline (the age in months) than a page in the screen to be shown. In other cases, the growth map screen may include a page-scroll button used to cause the growth map 21 to jump to the next page or a further page.

The growth map screen includes a view-favorite mark 23. When a user operates the view-favorite mark 23, the screen transitions to the favorite list screen shown in FIG. 3E indicating a list of favorites.

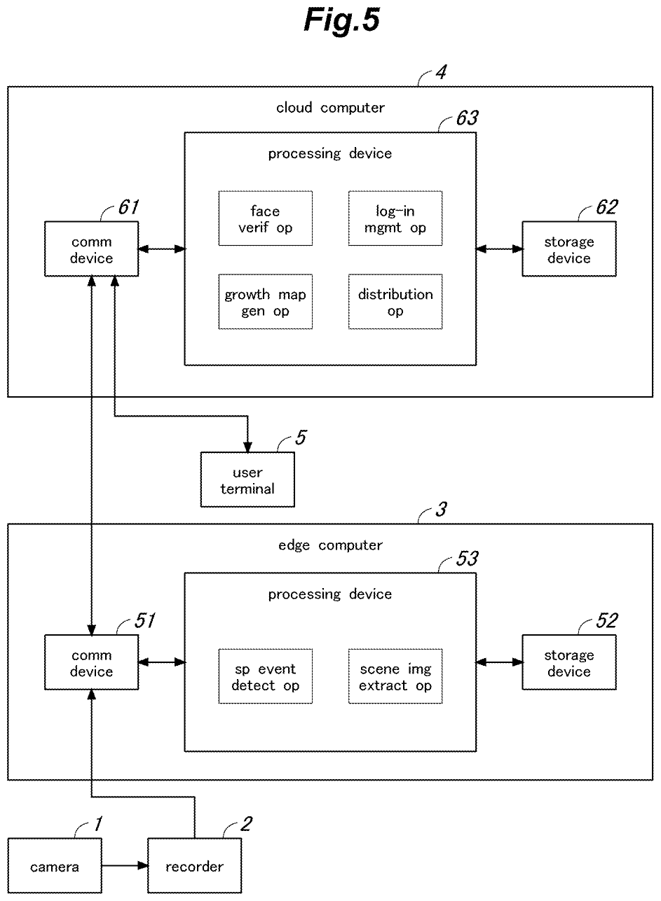

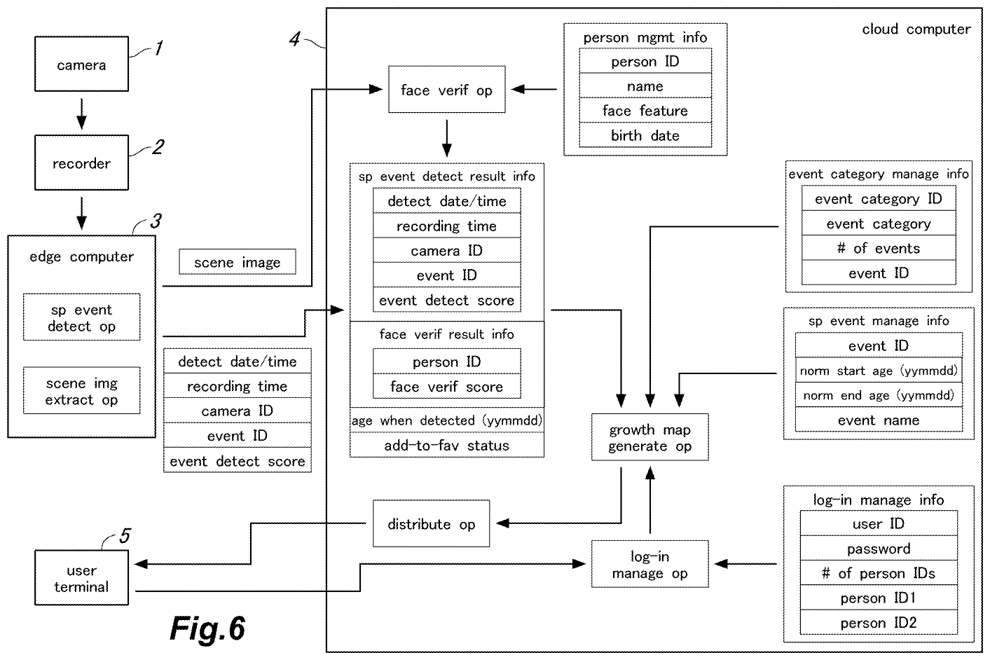

Next, schematic configurations of the edge computer 3 and the cloud computer 4 will be described. FIG. 5 is a block diagram showing schematic configurations of the edge computer 3 and the cloud computer 4. FIG. 6 is an explanatory diagram showing management information processed by the cloud computer 4.

The edge computer 3 includes a communication device 51, a storage device 52, and a processing device 53 (first processing device).

The communication device 51 communicates with the recorder 2 via a network. In the present embodiment, the communication device 51 receives images from the recorder 2, which stores the images that have been captured by the cameras 1. Furthermore, the communication device 51 communicates with the cloud computer 4 via the network. In the present embodiment, the communication device 51 transits images generated by the processing device 53 to the cloud computer 4.

The storage device 52 stores programs to be executed by the processing device 53 and other data.

The processing device 53 performs various processing operations for providing a lifelog by executing the programs stored in the storage device 52. In the present embodiment, the processing device 53 performs a specific event detection operation, a scene image extraction operation, and other operations.

In the specific event detection operation, the processing device 53 performs an image recognition operation on an image captured by a camera 1 and stored in the recorder 2, to thereby detect a specific event related to a level of growth of a child based on the result of the image recognition operation. The image recognition operation includes at least one of a body frame detection operation, an action recognition operation, and a facial expression estimation operation. The body frame detection operation can be used to recognize the motion of each part of a child. The action recognition operation can be used to recognize the action taken by a child. The facial expression estimation operation can be used to recognize facial expressions of a child, such as a child's smile.

It should be noted that the specific event detection operation can be performed using a recognition model constructed by machine learning technology (such as deep learning technology). When performing the image recognition operation, the system recognizes, in addition to a subject child, a person(s) and/or an item(s) around the child. For example, when detecting a child's shaking the rattle, the system also recognizes an object held in the child's hand in the specific event detection operation. When detecting a child's enjoying peek-a-boo, the system also recognizes a person (such as nursing staff) who is doing peek-a-boo.

In the scene image extraction operation, the processing device 53 extracts, based on the detection result of specific event detection operation, a scene image (moving image) including the detected specific event, from the images (moving images) captured by the cameras 1 and stored in the recorder 2.

The processing device 53 transmits a scene image extracted in the scene image extraction operation to the cloud computer 4. Furthermore, the processing device 53 transmits specific event detection result information to the cloud computer 4, the specific event detection result information including date and time of detection of a specific event, a moving image recording time of the scene image, the camera ID of a camera 1 that captured the scene image, the event ID of the detected specific event, and an event detection score (score indicating the certainty of the detected specific event).

In the scene image extraction operation, in addition to extracting a captured moving image showing a specific event, the processing device 53 may cut out a person image; that is, an image area of a subject person from the image captured by a camera 1. Specifically, the processing device 53 may cut out a detection frame of a person or a rectangular area including the detection frame.

The cloud computer 4 includes a communication device 61, a storage device 62, and a processing device 63 (second processing device).

The communication device 61 communicates with the edge computer 3 and the user terminal 5 via a network.

The storage device 62 stores programs to be executed by the processing device 63 and other data. The storage device 62 also stores scene images received from the edge computer 3. Furthermore, the storage device 62 stores management information. The storage device 62 may be provided with a large-capacity storage device such as a hard disk for storing scene images and management information.

The processing device 63 performs various processing operations for providing a lifelog by executing the programs stored in the storage device 62. In the present embodiment, the processing device 63 performs a face verification operation, a log-in (management) operation, a growth map generation operation, a distribution operation, and other operations.

In the face verification operation, the processing device 63 identifies a person appearing in a scene image received from the edge computer 3; that is, identifies a child whose specific event is detected. Specifically, the processing device 63 extracts face feature data of a child from the scene image, and compares the child's face feature data in the scene image with face feature data for each child included in person management information previously stored in the storage device 62, to thereby acquire a face verification score. Then, the processing device 63 identifies a person whose face verification score is equal to or greater than a predetermined threshold value, as the person (child) in the scene image. Based on the face verification operation result, the processing device 63 can associate the person in the scene image with the person management information for each person which was previously registered (person ID, name, and date of birth). Specifically, the processing device 63 acquires the person ID and the face verification score in the face verification operation and stores them in the storage device 62 as specific event detection result information.

In the log-in operation, the processing device 63 performs a log-in determination operation (user authentication) based on log-in management information stored in the storage device 62. When a user successfully logs in; that is, when the processing device 63 determines that a person who made a request for log-in is an authenticated user, the user is permitted to view the growth map 21 and scene images. The log-in management information includes the number of children (number of person IDs) and the children's person IDs for which the user is permitted to view the growth map 21 and scene images. Based on the log-in management information, the processing device 63 generates the person selection screen (see FIG. 3B).

In the growth map generation operation, the processing device 63 generates a growth map 21 for a child who is one of the children related to the logged-in user (parents and facility staff) and is selected by the user. In this operation, the processing device 63 creates a map image 28 (see FIG. 4) based on event category management information stored in the storage device 62. Specifically, the growth map is generated to include item rows 31, 32, 33 for the respective categories of specific events. Furthermore, the growth map is generated to include normal time range marks 35 (see FIG. 4) based on specific event management information (including standard start and end ages of children in months for each specific event) stored in the storage device 62. Then, based on detection date and time of each specific event and the date of birth of each person included in the specific event detection result information and the person management information, respectively, the processing device 63 calculates the age (year/month/date) of the child at the time of detection. The processing device 63 determines the location of each thumbnail 22 on the map image 28 based on the age of the child at the time of detection of a corresponding specific event.

In the distribution operation, in response to a user's instruction operation on the user terminal 5, the processing device 63 distributes the growth map 21 generated in the growth map generation operation to the user terminal 5, and causes the user terminal 5 to display the growth map 21. Moreover, in response to the user's instruction operation on the user terminal 5, the processing device 63 distributes a scene image (moving image) to the user terminal 5, and causes the user terminal 5 to reproduce the scene image.

Furthermore, the processing device 63 manages add-to-favorite statuses of specific events that have occurred for each child (add-to-favorite status management operation). In the add-to-favorite status management operation, the processing device 63 stores favorite list information in association with corresponding specific event detection result information and face verification result information, in the storage device 62. When a user operates the add-to-favorite mark 26 (see FIG. 3D), the processing device 63 performs an operation for adding a corresponding specific event to favorites. Furthermore, when a user operates the view-favorite mark 23 (see FIG. 3C), the processing device 63 displays the favorite list screen (FIG. 3E) based on the favorite list information on a list of favorites stored in the storage device 62.

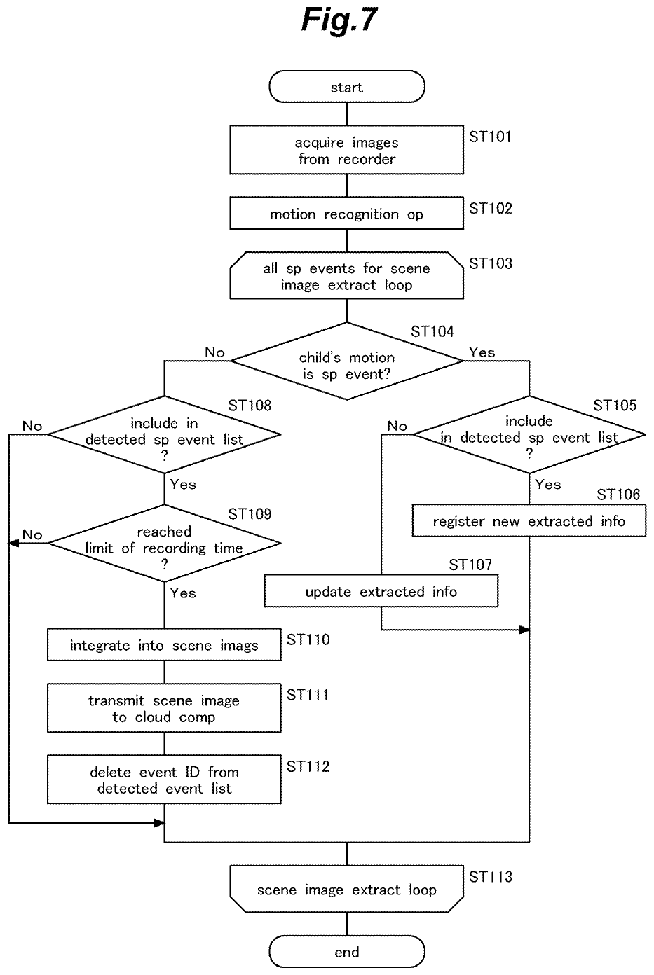

Next, processing operations performed by the edge computer 3 will be described. FIG. 7 is a flow chart showing a procedure of processing operations performed by the edge computer 3.

In the edge computer 3, the processing device 53 first acquires images captured by the cameras 1 and stored in the recorder 2 (ST101). The processing device 53 recognizes a child's motion from images captured by the cameras 1 and generates motion information representing the motion of each child (motion recognition operation) (ST102). Next, the processing device 53 performs a specific event detection operation and a scene image extraction operation for all specific events (ST103 to ST113). Specifically, the processing device 53 sequentially determines whether or not each frame of a captured image of each detected motion shows a corresponding specific event, and associates frames showing the specific event (usually several tens of frames continuously), with its event ID, and registers the frames in association with the event ID in a list of detected events. Then, when a captured image no longer shows the specific event, the processing device 53 determines, based on the event ID, whether extracted information related to the specific event was registered in the list of detected events in the past. Then, when the recording time of extracted information related to the specific event reaches the time limit, the processing device 53 performs an operation to integrate the extracted information (i.e., scene images) related to the specific event into a piece of extracted information.

In this operation, the processing device 53 first determines whether or not a child's motion recognized by the motion recognition operation corresponds to a certain specific event (motion determination operation) (ST104).

When the detected motion corresponds to a specific event; that is, when the specific event is detected (Yes in ST104), then the processing device 53 determines whether or not the detected specific event has an unregistered event ID; that is, whether or not the specific event is newly detected (ST105).

When the detected specific event has an unregistered event ID (Yes in ST105), the processing device 53 registers newly extracted information, which includes a scene image, in the list of detected events, the scene image being captured images showing the child's motion of the specific event (ST106). When the detected specific event has a registered event ID in the detected event list (No in ST105), the processing device 53 updates the extracted information with a new scene image (or adds the new scene image to the extracted information), the scene image being captured images showing the child's motion of the specific event (ST107).

When the detected motion does not correspond to any specific event; that is, when no specific event is detected (or a specific event ends) (No in ST104), then the processing device 53 determines whether or not that the specific event is a registered event in the list of detected events (ST108).

When the detected specific event is a registered event in the list (Yes in ST108), then the processing device 53 determines whether or not the recording time of extracted information; that is the total recording time of scene images (moving images) registered as extraction information has reached a predetermine time limit (recording time determination operation) (ST109).

When the recording time reaches the time limit (Yes in ST109), the processing device 53 then integrates a plurality of scene images registered as extraction information into a piece of extracted information (ST110). Then, the communication device 51 transmits the integrated scene image to the cloud computer 4 together with the event ID of the specific event shown in the scene image (ST111). Then, the processing device 53 deletes the extracted information associated with the event ID of the specific event from the list of detected events (ST112).

When the specific event is an unregistered event in the list of detected events (No in ST108), or when the recording time has not reached the time limit (No in ST109), the processing device 53 does not perform any operation for the specific event and the process proceeds to operations related to the next specific event.

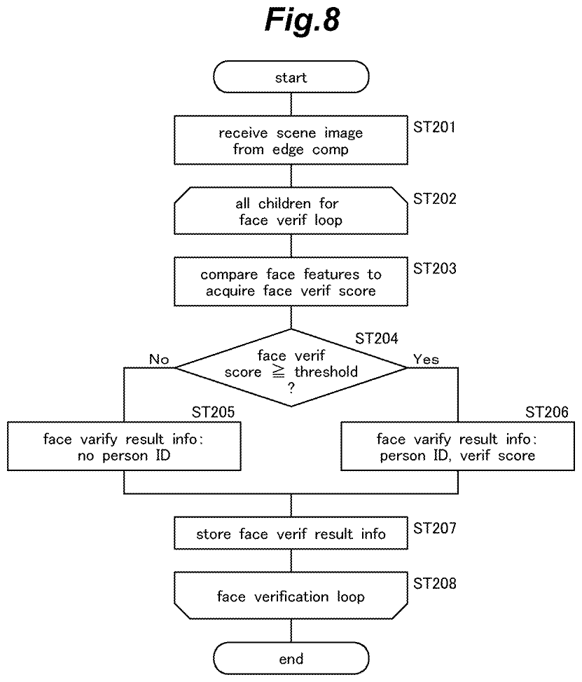

Next, a face verification operation performed at the cloud computer 4 will be described. FIG. 8 is a flow chart showing a procedure of the face verification operation performed at the cloud computer 4.

In the cloud computer 4, the communication device 61 first receives a scene image from the edge computer 3 (ST201). Next, the processing device 63 performs a face verification operation for every registered child, to thereby identify a child appearing in the scene image (ST202 to ST208).

In this operation, first, the processing device 63 extracts face feature data of a child from the scene image, and compares the face feature data of a child in the scene image with the pre-registered face feature data for each child previously stored in the storage device 62, to thereby acquire a face verification score (ST203). Then, the processing device 63 determines whether or not the face verification score is equal to or greater than a predetermined threshold value (face verification score determination) (ST204).

When the face verification score is equal to or greater than the threshold value (Yes in ST204), the processing device 63 generates face verification result information including the person ID and face verification score (ST206). When the face verification score is less than the threshold value (No in ST204), the processing device 63 determines that there is no relevant person in the scene image and generates face verification result information that does not include the person ID (ST205).

Next, the processing device 63 stores the face verification result information in the storage device 62 as specific event detection result information (ST207).

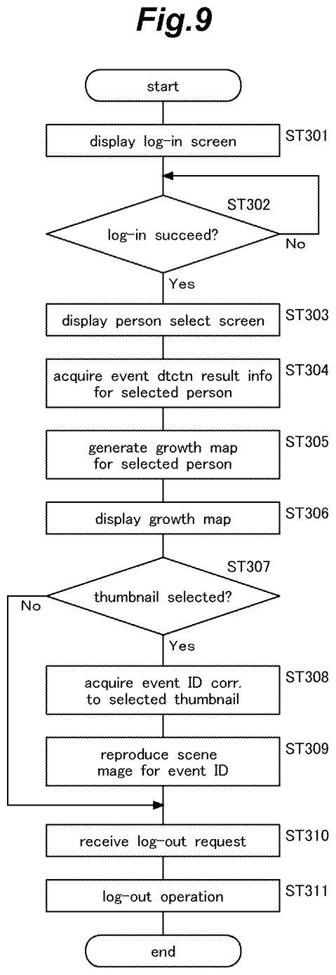

Next, the log-in operation, the growth map generation operation, and the distribution operation performed at the cloud computer 4 will be described. FIG. 9 is a flow chart showing a procedure of the log-in operation, the growth map generation operation, and the distribution operation performed at the cloud computer 4.

In the cloud computer 4, the processing device 63 first causes the user terminal 5 to display the log-in screen in response to a request for viewing from the user terminal 5 (ST301). Next, in the user terminal 5, when a user enters log-in information (ID and password) and operates on the screen to log-in, the communication device 61 receives a log-in request from the user terminal 5. Then the processing device 63 receives the log-in request and verifies the log-in information to determine whether or not the user can successfully log in; that is, whether or not the user is an authenticated user (ST302).

When the user successfully logs in (Yes in ST302), the processing device 63 causes the user terminal 5 to display the person selection screen (ST303). Next, when the user operates on the user terminal 5 to select a person (child), the processing device 63 acquires specific event detection result information for the selected person from the storage device 62 (ST304). Then, the processing device 63 generates a growth map 21 for the selected person based on the specific event detection result information for the selected person (ST305). Next, the processing device 63 distributes the growth map 21 to the user terminal 5 and causes the user terminal 5 to display the growth map (ST306).

When the user operates on the growth map screen displayed on the user terminal 5 to select a thumbnail 22 in the growth map (Yes in ST307), the processing device 63 determines the event ID of the specific event corresponding to the thumbnail 22 selected by the user. (ST308). Then, the processing device 63 distributes a scene image (moving image) corresponding to the event ID to the user terminal 5, and causes the user terminal 5 to reproduce the scene image (ST309).

When the user operates on the user terminal 5 to log out, the communication device 61 receives a log-out request from the user terminal 5 (ST310) and then the processing device 63 performs a log-out operation (ST311).

Specific embodiments of the present disclosure are described herein for illustrative purposes. However, the present disclosure is not limited to those specific embodiments, and various changes, substitutions, additions, and omissions may be made for features of the embodiments without departing from the scope of the invention. In addition, elements and features of the different embodiments may be combined with each other to yield an embodiment which is within the scope of the present disclosure.

### INDUSTRIAL APPLICABILITY

A lifelog providing system and a lifelog providing method according to the present disclosure achieve an effect of providing a user with a lifelog of a child that enables the user to systematically recognize a level of growth of the child, and are useful as a lifelog providing system and a lifelog providing method for providing a user with an image of a child captured by a camera in a childcare facility, as a lifelog.

### GLOSSARY

1 camera
2 recorder
3 edge computer
4 cloud computer
5 user terminal (user device)
21 growth map
22 thumbnail
23 view-favorite mark
25 map image
26 add-to-favorite mark
31, 32, 33 item row for specific event
34 column footer indicating age in months
35 normal time range mark
36 event detection mark
37 balloon
38 scroll button
51 communication device
52 storage device
53 processing device
61 communication device
62 storage device
63 processing device

1. A lifelog providing system in which at least one data processing device performs operations for providing a user with an image of a child captured by a camera in a facility, as a lifelog, wherein the at least one data processing device is configured to:
detect a specific event related to a level of growth of the child in images captured by the camera, by performing an image recognition operation;
extract a scene image including the detected specific event, from the images captured by the camera; and
generate, as a lifelog, a growth map in which a thumbnail of the scene image is overlaid on a reference map image, the reference map image including at least a timeline of child growth and an indicator showing a normal pace of growth of children for each specific event, such that the thumbnail of the scene image is located at a point in the reference map image corresponding to date and time of the detection of the specific event.

2. The lifelog providing system according to claim 1, further comprising:
an edge computer installed in the facility; and
a cloud computer connected to the edge computing device via a network;
wherein the at least one processing device comprises a first processing device provided in the edge computer and a second processing device provided in the cloud computer,
wherein the first processing device performs operations for detecting the specific event and extracting the scene image, and transmits the scene image to the cloud computer, and
wherein the second processing device generates the growth map based on the scene image received from the edge computer, and distributes the growth map to a user device.

3. The lifelog providing system according to claim 1, wherein the at least one processing device is configured to detect the specific event by performing the image recognition operation, wherein the image recognition operation comprises at least one of a body frame detection operation, an action recognition operation, and a facial expression estimation operation.

4. The lifelog providing system according to claim 1, wherein the at least one processing device is configured to, upon detecting a user's operation to select one of thumbnails in the growth map, cause a user device to display time information indicating date and time of occurrence of the specific event corresponding to the selected thumbnail.

5. The lifelog providing system according to claim 1, wherein the at least one processing device is configured to, upon detecting a user's operation to select one of thumbnails in the growth map, cause a user device to reproduce the scene image corresponding to the selected thumbnail.

6. The lifelog providing system according to claim 1, wherein the at least one processing device is configured to, upon detecting a user's add-to-favorite operation, add a selected specific event to favorites.

7. The lifelog providing system according to claim 1, wherein the at least one processing device is configured to, upon detecting a user's operation to view favorites, cause a user device to display a list of information on specific events in favorites.

8. A lifelog providing method in which at least one data processing device performs operations for providing a user with an image of a child captured by a camera in a facility, as a lifelog, wherein the at least one data processing device performs operations of:
detecting a specific event related to a level of growth of the child in images captured by the camera, by performing an image recognition operation;
extracting a scene image including the detected specific event, from the images captured by the camera; and
generating, as a lifelog, a growth map in which a thumbnail of the scene image is overlaid on a reference map image, the reference map image including at least a timeline of child growth and an indicator showing a normal pace of growth of children for each specific event, such that the thumbnail of the scene image is located at a point in the reference map image corresponding to date and time of the detection of the specific event.
